SEQUENCE LISTING (2 sequences)

SEQ ID NO: 1 - DNA, 885 bp, unknown
  CDS (1)..(885): bacterial
atggcacaggtgaaggccaacggcattaccctcgagtatgaagagcagggccatcgccaccatccgtccatgctcctcattatgggcctgggcggccagttaatcgactggcccgaggagttcatccgggggctggctgaacgaggcttccgggtaatctgtttcgacaaccgcgacgcggggctttcgacgaaacttgaaggcgtgaaaaaaccgaacattgcccgggtatttctcctggcgagcatgggcctaaagcccagggtgccttacaccctcgacgacatggccctggacaccgtggggctgatggatgccctgggcattgagagcacccacgtagttggcgtctccatgggcggcatgattgcgcagattctaggggcgaagcacggggagcgggtgaaatcccttaccctgatgattacctcctccggcaacccccgcatgccggcgcccaggccgcaggtgctgcaaaagtttatgcgggtgcccaagagcatggataaggaagagtggattaaatacaacttggagcttttaaccaccatcggcagccccgggttggaccgggagaagctggccttagacgtgaggaagagcatagagcggtgcctttgccccgaaggcacgcagcggcagctggcagccatcctgcagagcggcagcagggtgaagctgctccggcggatcgctgtccccaccctggtcatcagcggggcggaagatcccctcctgccgtaccagtgcggccgggacattgccgaccatatcccgggagcccgcttcgagctcatcgagggcatggggcacgacattcccgagcggcacatcccccggctgattgagctcatcgccgggcacgccgcggccgcggaagcttaa

SEQ ID NO: 2 - protein, 294 aa, unknown
  bacterial
MAQVKANGITLEYEEQGHRHHPSMLLIMGLGGQLIDWPEEFIRGLAERGFRVICFDNRDAGLSTKLEGVKKPNIARVFLLASMGLKPRVPYTLDDMALDTVGLMDALGIESTHVVGVSMGGMIAQILGAKHGERVKSLTLMITSSGNPRMPAPRPQVLQKFMRVPKSMDKEEWIKYNLELLTTIGSPGLDREKLALDVRKSIERCLCPEGTQRQLAAILQSGSRVKLLRRIAVPTLVISGAEDPLLPYQCGRDIADHIPGARFELIEGMGHDIPERHIPRLIELIAGHAAAAEA

(19) United States
(12) Patent Application Publication
(10) Pub. No.: US 2005/0153404 A1
(43) Pub. Date: Jul. 14, 2005

(54) Anti-kazlauskas lipases

(76) Inventors: Boris Bosch, Koln (DE); Ruth Meissner, Leverkusen (DE); Frank Berendes, Munster (DE); Rainhard Koch, Koln (DE)

Correspondence Address: LANXESS CORPORATION, 111 RIDC PARK WEST DRIVE, PITTSBURGH, PA, 15275-1112, US

(21) Appl. No.: 10/686,490

(22) Filed: Oct. 15, 2003

(30) Foreign Application Priority Data
Oct. 16, 2002 (DE) 10248166.0

(51) Int. Cl. C12P 13/06; C12P 21/06; C12P 7/62; C12Q 1/68; C07H 21/04; C12N 9/20

(52) U.S. Cl. 435/116; 435/198; 435/69.1; 435/320.1; 435/325; 536/23.2; 435/6; 435/135

(57) ABSTRACT

The present invention relates to polypeptides which exert the biological activity of lipases and which have an anti-Kazlauskas-rule selectivity, to host cells containing nucleic acids coding for these polypeptides, to the nucleic acids themselves, and to the vectors required for preparing the host cells of the invention. In a further aspect, the invention relates to a method for preparing enantiomer-enriched esters and alcohols, which is advantageously carried out in the presence of a racemization catalyst.

### BACKGROUND OF THE INVENTION

1. Field of the Invention

The present invention relates to polypeptides which exert the biological activity of lipases and which have an anti-Kazlauskas-rule selectivity, to host cells containing nucleic acids coding for these polypeptides, to the nucleic acids themselves, and to the vectors required for preparing the host cells of the invention. In a further aspect, the invention relates to a method for preparing enantiomer-enriched esters and alcohols, which is advantageously carried out in the presence of a racemization catalyst.

2. Brief Description of the Prior Art

Enantiomer-enriched alcohols are valuable compounds for preparing agrochemicals, medicaments, liquid crystals or intermediates thereof. They are frequently prepared using chiral transition metal catalysts. The art-related methods of preparing enantiomer-enriched alcohols and the attendant disadvantages of these methods are described hereunder. Mention must be made here in particular of reductions of ketones by transfer hydrogenation and hydrogenation (see also Noyori: Asymmetric Catalysis in Organic Synthesis, J. Wiley, 1994). Problems of the chemical-catalytic method are the frequently expensive catalysts, fluctuating noble metal prices and high demands on the purity of the substrates.

Enzymic kinetic racemate resolution of secondary alcohols via transesterification and its reversible reaction, the enzymic hydrolysis of esters of secondary alcohols, are important, industrially utilizable methods for preparing enantiomer-enriched alcohols. Here, one enantiomer of the racemic alcohol is converted selectively to the corresponding ester in the presence of a suitable acyl donor or one enantiomer of a racemic ester is selectively converted to the corresponding alcohol by hydrolysis of the ester bond. The transesterification may frequently be carried out in organic solvents and at high concentrations, due to the often-good solubility of the substrates. Furthermore, racemic mixtures of alcohols are usually readily obtainable as racemic esters. For these reasons, the enzyme-catalyzed transesterification is usually preferred to the enzyme-catalyzed hydrolytic cleavage of esters.

However, inherent disadvantages are that the maximally achievable yield of 50% and that the enantiomer excess may greatly decrease with increasing conversion, depending on enzyme selectivity. Another disadvantage is the fact that, according to the “Kazlauskas rule” (Kazlauskas et al., J. Org. Chem. 1991, 56, 2656), the hitherto known enzymes produce a particular, predictable three dimensional structure of the products, owing to the special structures of the active sites.

Accordingly, the enzyme-catalyzed transesterification of a racemic secondary alcohol usually proceeds according to the following scheme:

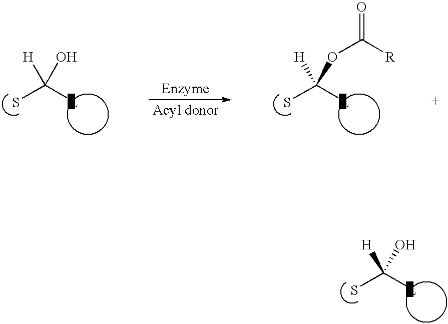

Here, S is a relatively small and L is a relatively large substituent. The figure displays absolute stereochemistry. With regard to prioritization of S and L and the determination of the absolute configuration (R or S), which ensues therefrom, the Cahn-Ingold-Prelog rule applies (CIP rule, see also J. March, Advanced Organic Chemistry, 4th Ed., 1992, Wiley Interscience, p. 109-115).

In addition, the Kazlauskas rule states that enzymes such as, in particular, lipases function in a particularly stereoselective fashion, if the spatial requirements of substituents S and L are very different.

There is therefore the need for providing polypeptides which enable chiral secondary alcohols to be transesterified to the corresponding esters with opposite selectivity with respect to the Kazlauskas rule with good enantioselectivities. Such enzymes will be referred to as anti-Kazlauskas lipases hereinbelow.

### SUMMARY OF THE INVENTION

Polypeptides have now been found which exert the biological activity of an anti-Kazlauskas lipase and which comprise an amino acid sequence which is at least 60%, preferably at least 80%, particularly preferably at least 90% and very particularly preferably at least 95% identical to an amino acid sequence according to SEQ ID NO: 2 across the section from amino acid 110 to amino acid 280 and preferably across their entire length. It is envisioned that a catalytic triad is formed by the amino acids 118, 243 and 271 according to SEQ ID NO: 2.

The term “polypeptides”, as used within the framework of the invention, refers both to short amino acid chains which are usually referred to as peptides, oligopeptides or oligomers and to longer amino acid chains which are usually referred to as proteins. It includes amino acid chains which may be modified either by natural processes such as posttranslational processing or by known chemical methods. Such modifications may occur at various sites and several times in a single polypeptide, such as, for example, on the peptide backbone, on the amino acid side chain, on the amino terminus and/or on the carboxy terminus. They include, for example, acetylations, acylations, ADP-ribosylations, amidations, covalent linkages to flavins, haeme moieties, nucleotides or nucleotide derivatives, lipids or lipid derivatives or phosphatidylinositol, cyclizations, disulphide bonds, demethylations, cystine bonds, formulations, gamma-carboxylations, glycosylations, hydroxylations, iodizations, methylations, myristoylations, oxidations, proteolytic processing, phosphorylations, selenoylations and tRNA-mediated additions of amino acids.

### DETAILED DESCRIPTION OF THE INVENTION

The polypeptides of the invention may be present in the form of mature proteins or as parts of larger proteins, for example as fusion proteins. Furthermore, they may have secretion or leader sequences, prosequences, sequences facilitating purification, such as multiple histidine radicals, or additional stabilizing amino acids.

The polypeptides of the invention need not be complete anti-Kazlauskas lipases but may also be only fragments thereof, as long as they still have at least 50% of the biological activity of the complete anti-Kazlauskas lipase. The invention likewise comprises polypeptides which exert at least 50% of the biological activity of the anti-Kazlauskas lipase with an amino acid sequence according to SEQ ID NO: 2.

The polypeptides of the invention may furthermore have deletions or amino acid substitutions in comparison with the corresponding region of natural anti-Kazlauskas lipases, as long as they at least still exert a biological activity of the complete anti-Kazlauskas lipase. Preference is given to conservative substitutions. Such conservative substitutions include variations, with one amino acid being replaced by another amino acid from the following group:
1. small aliphatic, non-polar or slightly polar residues: Ala, Ser, Thr, Pro and Gly;
2. polar, negatively charged residues and their amides: Asp, Asn, Glu and Gln;
3. polar, positively charged residues: His, Arg and Lys;
4. large aliphatic, non-polar residues: Met, Leu, Ile, Val und Cys; and
5. aromatic residues: Phe, Tyr and Trp.

The following list indicates preferred conservative substitutions:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

The degree of identity of the amino acid sequences is preferably determined with the aid of the BLASTP+BEAUTY program Version 2.0.4. (Altschul et al., Nucleic Acids Res., 25, 1997 3389-3402).

A particularly preferred polypeptide is the anti-Kazlauskas lipase with the amino acid sequence according to SEQ ID NO: 2.

The term “biological activity of a complete anti-Kazlauskas lipase”, as used within the framework of the invention, means the ability of converting substrates contrary to the Kazlauskas rule.

The invention furthermore comprises nucleic acids. The nucleic acids of the invention are in particular single-stranded or double-stranded deoxyribonucleic acids (DNA) or ribonucleic acids (RNA). Preferred embodiments are fragments of genomic DNA which may contain introns or cDNA.

The nucleic acids of the invention comprise particularly preferably a sequence selected from
a) the sequence according to SEQ ID NO: 1,
b) sequences coding for a polypeptide which comprises the amino acid sequence according to SEQ ID NO: 2,
c) partial sequences of at least 14 base pairs in length of the sequences defined in a) or b),
d) sequences hybridizing to the sequences defined in a) or b),
e) sequences which are at least 70%, preferably 85%, particularly preferably 90%, identical to the sequences defined in a),
f) sequences which are at least 70%, preferably 80%, particularly preferably 90%, identical to the sequences defined in b),
g) sequences which are complementary to the sequences defined in a) or b), and
h) sequences which code for the same amino acid sequence as the sequences defined under a) to f), owing to the degeneracy of the genetic code.

A very particularly preferred nucleic acid is a DNA molecule having the sequence according to SEQ ID NO: 1.

The term “to hybridize”, as used within the framework of the invention, describes the process in which a single-stranded nucleic acid molecule forms base pairs with a complementary strand. In this way it is possible to isolate, on the basis of the sequence information disclosed within the scope of the invention, for example, DNA fragments from other bacteria, which code for a polypeptide which has at least 50% of the activity of the inventive polypeptide having the amino acid sequence according to SEQ ID NO: 2.

The hybridization conditions are approximately calculated according to the following formula:

Melting temperature Tm=81.5° C.+16.6 log{c(Na⁺)]+0.41(% G+C))−500/n (Lottspeich and Zorbas, 1998, Bioanalytik, Spektrum Akademischer Verlag, Heidelberg, Berlin).

Here, c is the concentration and n the length in base pairs of the hybridizing sequence section. For a sequence >100 bp, the term 500/n is omitted. Washing is carried out with the highest stringency at a temperature of from 5 to 15° C. below Tm and an ionic strength of 15 mM Na⁺ (corresponds to 0.1×SSC). When using an RNA sample for hybridization, the melting point is higher by 10 to 15° C.

Preferred hybridization conditions are indicated below:

Hybridization solution: DIG Easy Hyb (Roche)

Hybridization temperature: 37° C., preferably 42° C. (DNA-DNA), 50° C. (DNA-RNA).

1st washing step: 2 times SSC, 2 times 5 min at room temperature;

2nd washing step: 2 times 15 min in 1×SSC, at 50° C.; preferably 0.5×SSC, at 65° C.; particularly preferably 0.2×SSC, at 65° C.

The degree of identity of the nucleic acids is preferably determined with the aid of the NCBI BLASTN program, Version 2.0.4.

The present invention furthermore relates to DNA constructs comprising a nucleic acid of the invention and a heterologous promoter.

The term “heterologous promoter”, as used within the framework of the invention, relates to a promoter whose properties are different from those of the promoter which controls expression of the gene in question in the source organism.

The selection of heterologous promoters depends on whether pro- or eukaryotic cells or cell-free systems are used for expression. Examples of heterologous promoters are the 35S promoter of cauliflower mosaic virus for plant cells, the alcohol dehydrogenase promoter for yeast cells, the lacZ-, T3-, T7- or SP6 promoters for prokaryotic cells or cell-free systems.

The present invention further relates to vectors which contain a nucleic acid of the invention or a DNA construct of the invention. Vectors which may be used are any phages, plasmids, phagemids, phasmids, cosmids, YACs, BACs, artificial chromosomes or particles suitable for particle bombardment, which are used in molecular-biological laboratories.

Examples of preferred vectors are the p4XXprom. vector series for yeast cells, pSPORT vectors (Invitrogen) for bacterial cells, lamdaZAP (Stratagene) for phages or the Gateway vectors (Invitrogen) for various expression systems in bacterial cells or baculovirus.

The present invention furthermore relates to host cells containing a nucleic acid of the invention, a DNA construct of the invention or a vector of the invention.

The term “host cell”, as used within the framework of the invention, refers to cells which do not naturally contain the nucleic acids of the invention.

Suitable host cells are both prokaryotic cells, preferably E. coli, and eukaryotic cells such as cells from Saccharomyces cerevisiae, Pichia pastoris, insects, plants, frog oocytes and mammalian cell lines.

The nucleic acids of the invention may be prepared in the usual manner. The nucleic acid molecules may be entirely chemically synthesized, for example. It is also possible to chemically synthesize short pieces of the nucleic acids of the invention and to radiolabel such oligonucleotides or label them with a fluorescent dye. The labelled oligonucleotides may then be used, for example, for screening cDNA banks generated starting from plant mRNA. Clones to which the labelled oligonucleotides hybridize are selected for isolating the corresponding DNA fragments. After characterization of the isolated DNA, the nucleic acids of the invention are obtained in a simple manner.

The nucleic acids of the invention may also be prepared by means of PCR methods using chemically synthesized oligonucleotides.

The term “oligonucleotide(s)”, as used within the framework of the invention, means DNA molecules comprising from 10 to 50 nucleotides, preferably from 15 to 30 nucleotides. They are chemically synthesized (Sambrook et al. (1989), Molecular Cloning, A Laboratory Manual, 2nd ed., Cold Spring Harbour Press) and may be used as probes.

I

The polypeptides of the invention may be obtained in various ways, for example by chemical methods such as the solid-phase method. In order to obtain larger amounts of protein, the use of recombinant methods is recommended. A cloned anti-Kazlauskas lipase gene or fragments thereof may be expressed in a manner known per se in a number of suitable host cells. For this purpose a nucleic acid of the invention, for example, is introduced into a host cell with the aid of known methods.

The integration of the cloned anti-Kazlauskas lipase gene into the host cell is within the scope of the present invention. Preferably the gene or fragments thereof are introduced into a plasmid, and the coding regions of the anti-Kazlauskas lipase gene or fragments thereof are functionally linked to a constitutive or inducible promoter.

The principal steps for preparing the recombinant anti-Kazlauskas lipase are:
1. Obtaining a natural, synthetic or semi-synthetic DNA coding for the anti-Kazlauskas lipase.
2. Introducing this DNA into an expression vector which is suitable for expressing the anti-Kazlauskas lipase, either alone or as fusion protein.
3. Transforming an appropriate, preferably prokaryotic host cell with this expression vector.
4. Growing this transformed host cell in a manner suitable for expressing the anti-Kazlauskas lipase.
5. Harvesting the cells and purifying anti-Kazlauskas lipase by means of suitable known methods.

All these steps may be carried out according to known procedures described for example in Sambrook et al. (1989), Molecular Cloning, A Laboratory Manual, 2nd ed., Cold Spring Harbour Press.

The coding regions of the anti-Kazlauskas lipase may be expressed in E. coli by the usual methods. Suitable expression systems for E. coli are commercially available, thus the expression vectors of the pET series, e.g. pET3a, pET23a, pET28a with His-Tag or pET32a with His-Tag for simple purification and thioredoxin infusion to increase the solubility of the expressed enzyme, and pGEX with glutathione synthetase fusion and also the pSPORT vectors, with the possibility to transfer the coding region of different vectors of the gateway system for various expression systems. The expression vectors are transformed, for example, into λ DE3-lysogenic E. coli strains, e.g. BL21(DE3), HMS 174(DE3) or AD494(DE3). After or during growing of the cells under standard conditions familiar to the skilled worker, expression may be induced using IPTG. Induction of the cells is usually followed by incubation at temperatures of from 18 to 37° C. for 3 to 24 hours. The cells may then be disrupted by sonification in disruption buffer (10 to 200 mM sodium phosphate, 100 to 500 mM NaCl, pH 5 to 8) or by incubation with lysis buffer. The expressed protein may be purified via precipitations or chromatographic methods.

The invention furthermore comprises a method for preparing compounds of the formula (I) 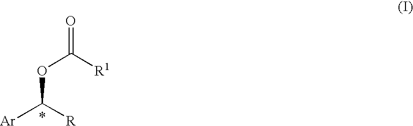
where the formula (I) indicates the absolute configuration of the product, and in which
* indicates a stereogenic carbon atom and
Ar is C₅-C₁₄-aryl and
R is cyano, C₁-C₁₂-alkyl, C₁-C₁₂-haloalkyl, C₅-C₁₁-arylalkyl or radicals of the formulae (IIa) to (IIf),
   A—B—D (IIa)
   A—D (IIb)
   A—SO₂—R³ (IIc)
   A—SO₃W (IId)
   A—COW (IIe)
   A—N₃ (IIf)
   in which, independently of one another,
A is absent or is a C₁-C₈-alkylene radical and
B is a carbonyl group and
D is R², OR², NHR³or N(R³)₂,
   where R² is C₁-C₈-alkyl, C₆-C₁₅-arylalkyl, C₁-C₈-haloalkyl or C₅-C₁₄-aryl and
   R³ is, in each case independently, C₁-C₈-alkyl, C₆-C₁₅-arylalkyl or C₆-C₁₄-aryl or N(R³)₂ together is a cyclic amino radical, and
   W is OH, NH₂, or OM, where M may be an alkali metal ion, half an equivalent of an alkaline earth metal ion, an ammonium ion or an organic ammonium ion, and
R¹ is C₁-C₁₂-alkyl, C₁-C₁₂-haloalkyl, C₅-C₁₁-arylalkyl, C₄-C₁₀-aryl, which method is characterized in that
stereoisomer mixtures of compounds of the formula (III) 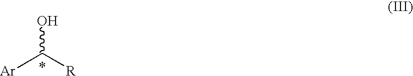
   in which
*, Ar and R are as defined in the formula (I)
are reacted with compounds of the formula (IV) 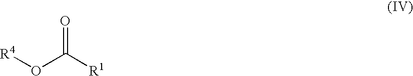
   in which
R¹ is as defined in the formula (I), and
R⁴ is C₁-C₁₂-alkyl, C₅-C₁₀-aryl, C₆-C₁₁-arylalkyl, C₂-C₁₂-alkenyl or C₁-C₁₂-haloalkyl
in the presence of polypeptides of the invention, which exert the biological activity of an anti-Kazlauskas lipase.

The abovementioned sequences, preferred ranges, parameters and explanations apply accordingly to the polypeptides usable in the method of the invention. The term “in the presence of polypeptides of the invention” comprises, for example, also the use of recombinant host cells as such or purified or non-purified cell lysates or cell preparations.

It is possible, within the scope of the invention, to combine any of the definitions of radicals, parameters and explanations, which are listed above and below and which are of a general nature or are mentioned in preferred ranges, with one another in any desired manner, i.e. also between the particular ranges and preferred ranges.

The term “stereoisomer-enriched” (“enantiomer-enriched”) in accordance with the invention comprises stereoisomerically pure (enantiomerically pure) compounds or random mixtures of stereoisomeric (enantiomeric) compounds, as long as the stereoisomeric (enantiomeric) compounds in these mixtures are not present in equal relative portions (e.g. Has racemate).

Alkyl and, respectively, alkoxy mean, in each case, independently, a straight-chain, cyclic, branched or unbranched alkyl and, respectively, alkylene and, respectively, alkoxy radical. The same applies to the non-aromatic part of an aryl alkyl radical.

C₁-C₄-Alkyl is, for example, methyl, ethyl, n-propyl, isopropyl, n-butyl, sec-butyl and tert-butyl, C₁-C₈-alkyl additionally is, for example, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-di-methylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, 1-ethyl-2-methylpropyl, n-heptyl and n-octyl, C₁-C₁₂-alkyl further additionally is, for example, n-nonyl, n-decyl and n-dodecyl.

C₁-C₄-Alkoxy is, for example, methoxy, ethoxy, n-propoxy, isopropoxy, n-butoxy, sec-butoxy and tert-butoxy, C₁-C₈-alkoxy additionally is n-pentoxy, 1-methylbutoxy, 2-methylbutoxy, 3-methylbutoxy, neopentoxy, 1-ethylpropoxy, cyclo-hexoxy, cyclo-pentoxy, n-hexoxy and n-octoxy, C₁-C₁₂-alkoxy further additionally is, for example, adamantoxy, the isomeric mentoxy radicals n-decoxy and n-dodecoxy.

Alkenyl is, within the framework of the invention, in each case independently a straight-chain, cyclic, branched or unbranched alkenyl radical having at least one olefinic double bond and bound via an olefinic carbon atom.

C₂-C₁₂-Alkenyl is, for example, vinyl, 1-propenyl, isopropenyl, 1-butenyl, 2-butenyl, 1-pentenyl, 2-pentenyl, 2-methyl-1-butenyl, 2-methyl-2-butenyl, 3-methyl-1-butenyl, 1-hexenyl, 1-heptenyl, 1-octenyl or 2-octenyl.

Haloalkyl is, within the framework of the invention, in each case independently a straight-chain, cyclic, branched or unbranched alkyl radical which is mono-, poly- or totally substituted with halogen atoms. The radicals which are totally substituted with fluorine are referred to as perfluoroalkyl.

For example, C₁-C₁₂-haloalkyl is, within the framework of the invention, trifluoro-methyl, 2,2,2-trifluoroethyl, chloromethyl, fluoromethyl, bromomethyl, 2-bromo-ethyl, 2-chloroethyl, nonafluorobutyl, n-perfluorooctyl and n-perfluorododecyl.

Aryl and Ar are, within the framework of the invention, for example carbocyclic aromatic radicals having from 6 to 14 backbone atoms, such as, for example, phenyl, naphthyl, phenanthrenyl and anthracenyl, or heteroaromatic radicals having from 5 to 14 backbone atoms, in which none, one, two or three backbone atoms per cycle are heteroatoms, but with at least one backbone atom of the entire molecule being a heteroatom, which heteroatoms are selected from the group consisting of nitrogen, sulphur or oxygen, such as preferably pyridinyl, thiophenyl, benzofuranyl, benzothiophenyl, dibenzofuranyl, dibenzothiophenyl, furanyl, indolyl, pyridazinyl, pyrazinyl, imidazolyl, pyrimidinyl and quinolinyl. Within the framework of the invention, information such as, for example, C₅ in the case of aryl radicals, refers to the number of carbon atoms of the aromatic backbone.

Furthermore, the carbocyclic aromatic radicals or heteroaromatic radicals may be substituted with up to 5 identical or different substituents per cycle. The substituents are, for example, preferably selected from the group consisting of fluoro, chloro, nitro, cyano, free or protected formyl, hydroxy, C₁-C₁₂-alkyl, C₁-C₁₂-haloalkyl, C₁-C₁₂-alkoxy, C₁-C₁₂-haloalkoxy, C₅-C₁₀-aryl such as, for example, phenyl, C₄-C₁₁-arylalkyl such as, for example, benzyl, di(C₁-C₁₂-alkyl)amino, (C₁-C₁₂-alkyl)amino, CO(C₁-C₁₂-alkyl), OCO(C₁-C₁₂-alkyl), NHCO(C₁-C₁₂-alkyl), N(C₁-C₆-alkyl)CO(C₁-C₁₂-alkyl), CO(C₃-C₁₂-aryl), OCO(C₃-C₁₂-aryl), NHCO(C₃-C₁₂-aryl), N(C₁-C₆-alkyl)CO(C₃-C₁₂-aryl), COO-(C₁-C₁₂)-alkyl, COO-(C₃-C₁₂)-aryl, CON(C₁-C₁₂-alkyl)₂ or CONH(C₁-C₁₂-alkyl) CO₂M, CONH₂, SO₂NH₂, SO₂N(C₁-C₁₂-alkyl)₂, SO₃M, M being in each case unsubstituted or substituted ammonium, lithium, sodium, potassium or caesium.

Analogously, the definition and the preferred ranges apply within the scope of the invention also to the aromatic part of an arylalkyl radical.

Ar is preferably phenyl, naphthyl, pyridinyl, oxazolyl, thiophenyl, furanyl, benzofuranyl, benzothiophenyl, dibenzofuranyl, dibenzothiophenyl, indolyl, pyridazinyl, pyrazinyl, imidazolyl, pyrimidinyl and quinolinyl, which may be further substituted with none, one, two, three or four radicals per cycle, which radicals are selected from the group consisting of hydroxy, fluoro, chloro, bromo, nitro, cyano, C₁-C₈-alkyl, C₁-C₈-perfluoroalkyl, C₁-C₈-alkoxy, di(C₁-C₄-alkyl)amino, COO(C₁-C₄-alkyl), NHCO(C₁-C₄-alkyl), CON(C₁-C₄-alkyl)₂, COO(C₆-C₁₁-arylalkyl), C₆-C₁₁-arylalkyl or C₅-C₁₀-aryl.
Ar is particularly preferably phenyl which may be substituted with none, one or two radicals which are selected from the group consisting of hydroxy, fluoro, chloro, bromo, methyl, ethyl, isopropyl, methoxy, benzyloxy, nitro, cyano, trifluoromethyl, phenyl, dimethylamino and N-acetylamino.
Ar is very particularly preferably phenyl and p-chlorophenyl.
R is preferably methyl, ethyl, butyl, isopropyl, benzyl, chloromethyl, bromomethyl and trifluoromethyl.
R¹ is preferably methyl, n-butyl and isopropyl.
R⁴ is particularly preferably ethyl, vinyl, isopropenyl, isopropyl and p-chloro-benzoyl.

Particularly preferred compounds of the formula (III) are p-chlorobenzoyl acetate, ethyl acetate, isopropyl butyrate, isopropyl acetate and isopropenyl acetate, trifluoroethyl butyrate.

Preferred stereoisomer mixtures used are enantiomer mixtures of compounds of the formula (II). The ratio of (R)- to (S)-configured enantiomers of the compounds of the formula (II) in the starting mixture may be, for example, from 5:95 to 95:5, preferably from 30:70 to 70:30. Very particular preference is given to using a racemic mixture.

Examples of solvents suitable for the method of the invention are: aliphatic and aromatic, unhalogenated and halogenated hydrocarbons such as, for example, chlorobenzene, xylene, toluene, benzene, hexane and dichloromethane.

The starting concentrations of compounds of the formula (II) may be, for example, between 0.01 and 2 mol/l, 0.1 to 1 mol/l being preferred.

The reaction temperature may be, for example, from 5 to 120° C., preferably from 20 to 100° C., particularly preferably 40 to 80° C., particularly preferably 60 to 80° C.

The pressure during the reaction may be, for example, from 0.01 to 10 bar, particularly preferably from 0.05 to 1.2 bar and very particularly preferably from 0.05 to 0.8 bar.

In a preferred embodiment, the reduced pressure enables most of the volatile reaction product of the compounds of the formula (III) to be removed from the reaction mixture by distillation.

The reaction times are, for example, from 6 to 96 hours, preferably from 12 to 24 hours.

In the conversion of racemic phenethyl alcohol, for example, the (S)-configured alcohol of the formula (II) is converted with increasing reaction time in a manner according to the invention to the (S)-configured ester of the formula (II), the (R)-configured alcohol of the formula (II) being concentrated in the reaction mixture.

The reaction is usually stopped when conversion of the stereoisomer mixture used at the start reaches from 60 to 100%, preferably from 80 to 90%, of the percentage of alcohol of the formula (II), i.e. with a conversion of from 30 to 50%, preferably from 40 to 49%, in racemic mixtures.

The compounds of the formula (I) may then be removed from the unreacted compounds of the formula (II) in a manner known per se, for example by distillation and/or chromatography.

In a particularly preferred embodiment, however, the method of the invention is carried out in the presence of a catalyst which racemizes stereoisomer-enriched mixtures of compounds of the formula (II).

In this way, a concentration of the non-acylated alcohol of the formula (II) is substantially suppressed by continuous racemization, and in the end, in the optimal case, the entire amount of the stereoisomer mixture of alcohols of the formula (II) used is converted to the ester. In this way it is possible to avoid separation of alcohol and ester.

Preferred catalysts here are those containing ruthenium complexes. Particularly preferred catalysts are those which contain
a) ruthenium complexes of formula (IV), 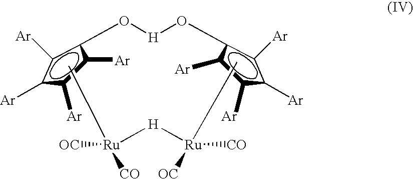
   in which
Ar is, in each case independently preferably identical to, phenyl which is unsubstituted or mono-, di-, tri- or tetrasubstituted with C₁-C₄-alkyl, phenyl being still further preferred,
and/or
b) ruthenium complexes of the formula (V),
   [RuX₂(Aren)]₂ (V)
   in which
Aren is a coordinated aromatic compound having from 6 to 12 ring carbons, which may furthermore be substituted with up to 6 radicals which are, in each case independently of one another, selected from the group consisting of C₁-C₈-alkyl, benzyl and phenyl and
X is chlorine, bromine or iodine, preferably chlorine
and/or
c) ruthenium complexes of the formula (VI),
   [RuX₂(Aren) {(VII)}] (VI)
   where
Aren and X are in each case as defined in formula (V) and (VII) is secondary or tertiary diamines, monoacylated or monosulphonated diamines, amino alcohols, amino acids and amino acid amides.

In the formula (V) arene is preferably benzene or naphthalene which may be substituted with up to 6 radicals which are, in each case independently of one another, selected from the group consisting of methyl, ethyl, n-propyl, isopropyl and tert-butyl.

Arene is preferably mesitylene, cumene or benzene.

Particularly preferred compounds of the formula (V) are benzenedichlororuthenium dimer, mesitylenedichlororuthenium dimer and cumenedichlororuthenium dimer.

In the formula (V), (VII) preferably represents compounds of the formula (VII) 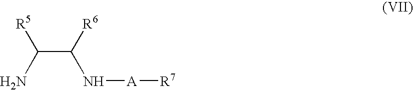
in which
R ⁵and R⁶ are, in each case independently of one another, hydrogen, C₁-C₂₀-alkyl, C₄-C₁₅-aryl or C₅-C₁₆-arylalkyl, for example, or R⁵ and R⁶ together are a straight-chain or branched C₃-C₁₂-alkylene radical, and
R⁷ is C₁-C₂₀-alkyl, C₁-C₂₀-fluoroalkyl or C₄-C₁₅-aryl, and
A is SO₂ or CO, preferably SO₂.

Preferably, R⁵ and R⁶ are in each case identical to phenyl or are together straight-chain C₃-C₈-alkylene such as, for example, 1,3-propylene or 1,4-butylene, and particularly preferably R and R are in each case identical to phenyl.
R⁷ is preferably C₁-C₄-alkyl, C₁-C₄-fluoroalkyl, phenyl or naphthyl which may be further substituted with none, one, two, three, four or five radicals selected from the group consisting of C₁-C₄-alkyl, C₁-C₄-alkoxy, C₁-C₄-fluoroalkyl, fluoro and chloro, particularly preferably methyl, trifluoromethyl, pentafluoroethyl, nonafluorobutyl, phenyl, p-tolyl, p-ethylphenyl, p-anisyl, p-ethoxyphenyl, p-chlorophenyl, 2,4,6-trimethylphenyl, 2,4,6-triisopropylphenyl, p-fluorophenyl, pentafluorophenyl, and naphthyl, p-tolyl, phenyl and naphthyl being still further preferred.

The catalyst is preferably used in the presence of a base such as, for example, amines of the NHₙ(C₁-C₁₈-alkyl)₍₃₋ₙ₎ type, where n may be one, two or three, preferably three. A particularly preferred amine is triethylamine. Furthermore it is possible to use alkali metal or alkaline earth metal carbonates as bases.

The molar amount of ruthenium may be, for example, 0.01 to 5 mol %, based on the substrate used, preferably 0.1 to 2 mol %, very particularly preferably 0.2 to 1 mol %.

It is advantageous, albeit not absolutely necessary, to carry out the reaction in an oxygen-free atmosphere.

It is possible to obtain in a manner of the invention stereoisomer-enriched esters of the formula (I) which may be purified in a manner known per se, for example by distillation.

Furthermore it is possible to convert the compounds of the formula (I) via saponification which may be carried out, for example, as acidic or basic saponification in a manner known per se to compounds of the formula (IIa) 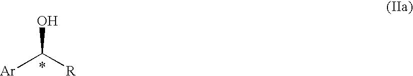
in which
*, Ar and R are as defined in the formula (I), including their preferred ranges.

The invention likewise comprises a method for preparing compounds of the formula (IIa) via hydrolysis of stereoisomer mixtures of compounds of the formula (Ia) 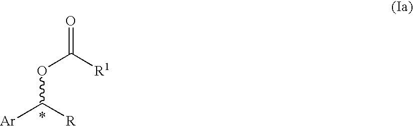
in which
Ar, R and R¹ are as defined in the formula (I), including their preferred ranges, and
indicates an (S)- or (R)-configured carbon atom
   in the presence of the polypeptides of the invention; however, this method is not a preferred preparation method.

The compounds of the formulae (I) and (IIa), which can be prepared according to the invention, are particularly suitable for application in a method for preparing medicaments or agrochemicals and for preparing and as intermediates of medica-ments or agrochemicals.

The advantage of the invention is the possibility of producing from a stereoisomer mixture of chiral alcohols selectively the opposite enantiomer of the corresponding ester in comparison with known enzymic racemate resolutions by using the “anti-Kazlauskas lipases” of the invention.

In addition, it is possible to prepare enantiomer-enriched esters with very high yields and very good enantioselectivities by combining the lipases of the invention with a racemization catalyst. Furthermore, good enantioselectivity is also observed if the spatial requirements of the different substituents R and S are relatively low, this being an advantage compared to hitherto known enzymes which can only induce high enantioselectivities with large differences between the substituent L and S.

### EXAMPLES

### Example 1

A total of 275 enzymes from genebanks were characterized in conversion experiments and tested for their S-selectivity towards stereoisomer mixtures of chiral alcohols.

Genebanks were prepared by isolating DNAs from various environmental samples and subsequently cloning them into appropriate vector systems These genebanks are then transformed into host cells such as, for example, Escherichia coli, where they express the heterologous DNA (Sambrook et al. (1989), Molecular Cloning, A Laboratory Manual, 2nd ed., Cold Spring Harbour Press. Using suitable screening methods, recombinant clones having the desired enzymic activity can be identified. This method makes it possible to access a multiplicity of the enzymes found in nature.

In order to test for a possible use in dynamically kinetic racemate resolution, the etherification reaction was carried out in an organic solvent at high temperatures. The recombinant E. coli cultures were grown by methods known to the skilled worker with the addition of inducer (IPTG) at 37° C. overnight and removed by centrifugation on the next morning. The cells are lysed by resuspending the cell pellets in lysis buffer (20 mM ammonium formate pH 6.5, 0.5% lysozyme, 0.5% dodecyl β-D maltoside), followed by incubation with shaking at 37° C. for 3 to 4 hours. Coarse cell debris was then again removed by centrifugation and the supernatant was lyophilised. The cell lyophilisates are used as catalysts for the reactions.

The reactions were carried out in 1 ml of toluene (dried) under the following reaction conditions:
one spatula-tip of lyophilisate
0.2 M substrate
0.6 M vinyl acetate
4 hours at 60° C. or 80° C.

After the reaction, the samples are measured with chiral GC and the results are analysed.

In the experiments an anti-Kazlauskas lipase having the amino acid sequence according to SEQ ID NO: 2 was identified (BLASTP+BEAUTY program Version 2.0.4. (Altschul et al., Nucleic Acids Res., 25, 1997 3389-3402).

General Protocol for the Racemate Resolution of Phenylalkanols:

The anti-Kazlauskas lipase in 4 ml of toluene is initially introduced into a reaction tube, the vessel is flushed with argon and the alcohol, the acylating agent and, where appropriate, the racemization catalyst are added. The mixture is heated to the desired temperature and stirred for the time indicated.

The results of the acylation catalyzed by the anti-Kazlauskas lipase are listed in Table 1.

TABLE 1

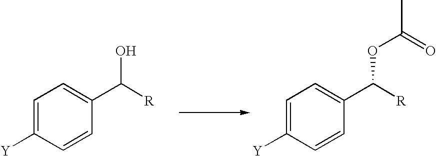

| | Lyophilisate | Acylating | | | | T | | | ee |
|---|---|---|---|---|---|---|---|---|---|
| Example | [% by wt.] | agent | Y | R | Cat. | [° C.] | t [h] | Y [%] | (Ester[%]) |
| 2 | 10 | VAc | H | Me | − | 80 | 16 | 11.7 | 44.4 (S) |
| 3 | 10 | VAc | H | Me | − | 80 | 40 | 16.3 | 38 (S) |
| 4 | 10 | ClBA | H | Me | − | 80 | 40 | 11.6 | 45 (S) |
| 5 | 10 | ClBA | H | Me | + | 80 | 16 | 27.3 | 34 (S) |
| 6 | 5 | ClBA | H | Et | + | 80 | 16 | 24 | n.d. |
| 7 | 5 | ClBA | H | Et | + | 80 | 40 | 31.9 | 41.8 (S) |
| 8 | 5 | ClBA | H | Et | − | 80 | 40 | 3.9 | 56.6 (S) |
| 9 | 10 | ClBA | H | i-Pr | + | 80 | 40 | 14.7 | 80.4 (S) |
| 10 | 10 | ClBA | Cl | Me | − | 80 | 80 | 3.0 | 45 (S) |
| 11 | 10 | ClBA | H | | + | 80 | 40 | <1 | 0 |
| (f. comp.) | | | | | | | | | |

Notes regarding the table
Acylating agent:
Vac = vinyl acetate (3 eq), ClBA = chlorobenzoyl acetate (3 eq),
Cat. = catalyst (2 mol% [(C₄Ph₄COHOCC₄Ph₄)(μ-H)][(CO)₄RU₂]each)
f. comp.: comparative example: Candida Antarctica 435 enzyme (Novo Nordisk).

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. Nucleic acids coding for polypeptides which exert the biological activity of an anti-Kazlauskas lipase and comprise an amino acid sequence according to SEQ ID NO: 2.

2. Nucleic acids according to claim 1, characterized in that they are single-stranded or double-stranded deoxyribonucleic acids (DNA) or ribonucleic acids (RNA).

3. Nucleic acids according to claim 1, characterized in that they are fragments of genomic DNA or cDNA.

4. Nucleic acids according to claim 1, comprising a sequence selected from
a) the sequence according to SEQ ID NO: 1,
b) sequences coding for a polypeptide which comprises the amino acid sequence according to SEQ ID NO: 2,
c) partial sequences of at least 14 base pairs in length of the sequences defined in a) or b),
d) sequences hybridizing to the sequences defined in a) or b),
e) sequences which are at least 70% identical to the sequences defined in a),
f) sequences which are at least 70% identical to the sequences defined in b),
g) sequences which are complementary to the sequences defined in a) or b), and
h) sequences which code for the same amino acid sequence as the sequences defined under a) to f), owing to the degeneracy of the genetic code.

5. DNA construct comprising a nucleic acid according to claim 1 and a heterologous promoter.

6. Vector comprising a nucleic acid according to claim 1 or a DNA construct comprising said nucleic acid and a heterologous promoter.

7. Host cell containing a nucleic acid according to claim 1, a DNA construct comprising said nucleic acid and a heterologous promoter, or a vector comprising said nucleic acid or said DNA construct and a heterologous promoter.

8. Host cell according to claim 7, characterized in that it is a prokaryotic cell.

9. Method for preparing a nucleic acid according to claim 1, comprising
(i) chemically synthesizing the nucleic acid or
(ii) a) chemically synthesizing oligonucleotides,
b) radiolabelling or labelling the oligonucleotides with a fluorescent dye,
c) hybridizing the labelled oligonucleotides to DNA of a genomic or cDNA bank generated, starting from plant mRNA or genomic DNA,
d) selecting clones which the labelled oligonucleotides hybridize and
e) isolating the hybridized DNA, or
(iii) chemically synthesizing oligonucleotides and amplifying them by means of PCR.

10. Polypeptide having the biological activity of an anti-Kazlauskas lipase, which is encoded by a nucleic acid according to claim 1.

11. Polypeptide having the biological activity of an anti-Kazlauskas lipase, which comprises an amino acid sequence which is at least 60% identical to an amino acid sequence according to SEQ ID NO: 2 across at least the section from amino acid 110 to amino acid 280.

12. Method for preparing a polypeptide according to claim 10 characterized in that
the preparation is carried out using chemical methods or
host cells grown under conditions allowing expression of a nucleic acid, the grown host cells are harvested and the polypeptide is recovered therefrom and, where appropriate, purified.

13. Method for preparing compounds of the formula (I) 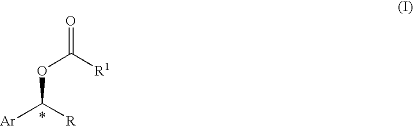
where the formula (I) indicates the absolute configuration of the product, and in which
* indicates a stereogenic carbon atom and
Ar is C₅-C₁₄-aryl and
R is cyano, C₁-C₁₂-alkyl, C₁-C₁₂-haloalkyl, C₅-C₁₁-arylalkyl or radicals of the formulae (IIa) to (IIf),
A—B—D (IIa)
A—D (IIb)
A—SO₂—R³ (IIc)
A—SO₃W (IId)
A—COW (IIe)
A—N₃ (IIf)
in which, independently of one another,
A is absent or is a C₁-C₈-alkylene radical and
B is a carbonyl group and
D is R², OR², NHR³or N(R³)₂,
where R² is C₁-C₈-alkyl, C₆-C₁₅-arylalkyl, C₁-C₈-haloalkyl or C₅-C₁₄-aryl and
R³ is, in each case independently, C₁-C₈-alkyl, C₆-C₁₅-arylalkyl or C₆-C₁₄-aryl or N(R³)₂ together is a cyclic amino radical, and
W is OH, NH₂, or OM, where M may be an alkali metal ion, half an equivalent of an alkaline earth metal ion, an ammonium ion or an organic ammonium ion, and
R¹ is C₁-C₁₂-alkyl, C₁-C₁₂-haloalkyl, C₅-C₁₁-arylalkyl, C₄-C₁₀-aryl,
comprising reacting
stereoisomer mixtures of compounds of the formula (III) 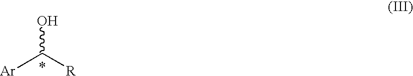
in which
*, Ar and R are as defined in the formula (I)
with compounds of the formula (IV) 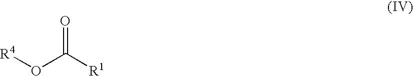
in which
R¹ is as defined in the formula (I), and
R⁴ is C₁-C₁₂-alkyl, C₄-C₁₀-aryl, C₅-C₁₁-arylalkyl, C₂-C₈-alkenyl or C₁-C₁₂-haloalkyl
in the presence of polypeptides of the invention, which exert the biological activity of an anti-Kazlauskas lipase.

14. Method according to claim 13, characterized in that Ar is phenyl, naphthyl, pyridinyl, oxazolyl, thiophenyl, furanyl, benzofuranyl, benzothiophenyl, dibenzofuranyl, dibenzothiophenyl, indolyl, pyridazinyl, pyrazinyl, imidazolyl, pyrimidinyl or quinolinyl, which is optionally unsubstituted or further substituted with one, two or three or four radicals per cycle, the said radicals being selected from the group consisting of hydroxy, fluoro, chloro, bromo, nitro, cyano, C₁-C₈-alkyl, C₁-C₈-perfluoroalkyl, C₁-C₈-alkoxy, di(C₁-C₄-alkyl)amino, COO(C₁-C₄-alkyl), NHCO(C₁-C₄-alkyl), CON(C₁-C₄-alkyl)₂, COO(C₆-C₁₁-arylalkyl), C₆-C₁₁-arylalkyl or C₅-C₁₀-aryl.

15. Method according to claim 13, characterized in that the compound of the formula (III) used is p-chlorobenzoyl acetate, ethyl acetate, isopropyl butyrate, isopropyl acetate, isopropentyl acetate or trifluoroethyl butyrate.

16. Method according to claim 13, characterized in that the stereoisomer mixtures used are racemic mixtures of compounds of the formula (II).

17. Method according to claim 13, characterized in that the reaction is stopped when conversion of the stereoisomer mixture employed at the start reaches 60 to 100% of the percentage of (S)-configured alcohol of the formula (II).

18. Method according to claim 13, characterized in that the method is carried out in the presence of a catalyst which racemizes the stereoisomer-enriched mixtures of compounds of the formula (II).

19. Method according to claim 18, characterized in that the catalysts used are those containing ruthenium complexes.

20. Method according to claim 18, characterized in that the catalysts used are those containing
a) ruthenium complexes of formula (IV), 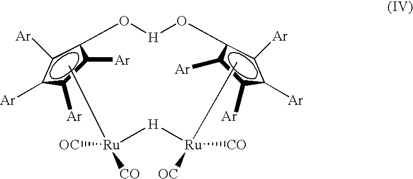
in which
Ar is, in each case independently, phenyl which is unsubstituted or mono-, di-, tri- or tetrasubstituted with C₁-C₄-alkyl,
and/or
b) ruthenium complexes of the formula (V),
[RuX₂(Aren)]₂ (V)
in which
Aren is a coordinated aromatic compound having from 6 to 12 ring carbons, which is optionally furthermore substituted with up to 6 radicals which are, in each case independently of one another, selected from the group consisting of C₁-C₈-alkyl, benzyl and phenyl and
X is chlorine, bromine or iodine, preferably chlorine
and/or
c) ruthenium complexes of the formula (VI),
[RuX₂(Aren){(VII)}] (VI)
where
Aren and X are in each case as defined in formula (V) and (VII) is secondary or tertiary diamines, monoacylated or monosulphonated diamines, amino alcohols, amino acids and amino acid amides.

21. Method according to claim 20, characterized in that in the formula (VI) (VII) represents compounds of the formula (VII), 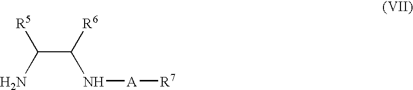
in which
R⁵ and R⁶are, in each case independently of one another, hydrogen, C₁-C₂₀-alkyl, C₄-C₁₅-aryl or C₅-C₁₆-arylalkyl, or R⁵ and R⁶ together are a straight-chain or branched C₃-C₁₂-alkylene radical, and
R⁷ is C₁-C₂₀-alkyl, C₁-C₂₀-fluoroalkyl or C₄-C₁₅-aryl, and
A is SO₂ or CO.

22. Method according to claim 18, characterized in that the catalyst is used in the presence of a base.

23. Method according to claim 18, characterized in that in a further step the compounds of the formula (I) are saponified to give compounds of the formula (IIa) 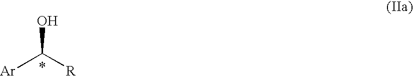
in which
*, Ar and R are as defined in the formula (I) in claim 18.

24. Method for preparing compounds of the formula (IIa) 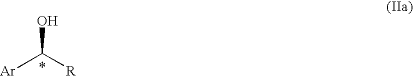
in which
Ar and R are as defined in the formula (I) in claim 13, and
* indicates an (S)-stereogenic carbon atom in its absolute configuration,
comprising hydrolyzing
stereoisomer mixtures of the formula (Ia) 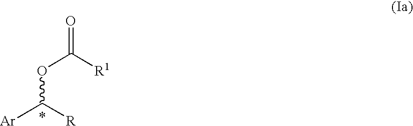
in which
Ar, R and R¹ are as defined in the formula (I), and
indicates an (S)-stereogenic carbon atom in the presence of polypeptides according to claim 10.

25. A process for preparing medicaments or agrochemicals or for the preparation of intermediates of medicaments or agrochemicals comprising incorporating the compounds prepared according to claim 13.

26. Method for preparing compounds of the formula (IIa) 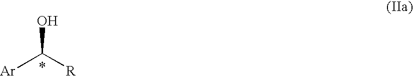
in which
Ar and R are as defined in the formula (I) in claim 13, and
* indicates an (S)-stereogenic carbon atom in its absolute configuration,
comprising hydrolyzing
stereoisomer mixtures of the formula (Ia) 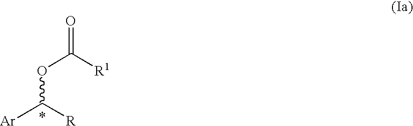
in which
Ar, R and R¹ are as defined in the formula (I), and
* indicates an (S)-stereogenic carbon atom
in the presence of polypeptides according to claim 11.
